(19) United States
(12) Patent Application Publication
(10) Pub. No.: US 2009/0173799 A1
(43) Pub. Date: Jul. 9, 2009

(54) Volatile Liquid Emitting Device

(75) Inventors: Colin Litten-Brown, Bracknell Berkshire (GB); Guy Edward Naish, Bicester Oxfordshire (GB); Kishen Gohil, Surrey (GB)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 12/299,632

(22) PCT Filed: Apr. 27, 2007
(86) PCT No.: PCT/CH07/00208

§ 371 Date: Nov. 21, 2008

(30) Foreign Application Priority Data
May 8, 2006 (GB) 0608919.7

(51) Int. Cl. A61L 9/04

(52) U.S. Cl. 239/6; 239/44

(57) ABSTRACT

A device adapted to disseminate into an atmosphere in sequence a plurality of active volatile liquids, such as fragrances, comprising a plurality of individual liquid reservoirs, a dissemination element associated with each reservoir, a central source of forced air current and one air channel per reservoir leading from an entrance at the central source to each dissemination element, air current being provided to one channel at a time by means of an air-tight rotatable barrier comprising a plurality of apertures, the spacing of these apertures being adapted to close all but one entrance at any given time.

The device may be provided as a simple plug-in type and substantially overcomes the problem of “habituation” encountered with single fragrance air fresheners.

This invention relates to devices for disseminating a plurality of volatile liquids into an atmosphere.

Devices for disseminating into an atmosphere an active volatile liquid are well known to the art. By “active” is meant a liquid whose presence in the atmosphere is desired for the performance of some function—typical examples include a fragrance, a disinfectant, an odour masking agent or a fungicide. A wide variety of such devices is commercially available. While most of these rely on evaporation alone, many have assisted dissemination, in that the evaporation is augmented by a heating element, a forced air current or both. These are generally powered by electricity, supplied by internal or external batteries, solar cells or mains power. One common and particularly desirable type of augmented evaporation device is the so-called “plug-in” type, a relatively small device that comprises, in a single unit, reservoir, dissemination element (such as a porous wick), electrically-driven augmentation element (such as heater and/or fan), transformer and power outlet pins, allowing it to be plugged directly into a power outlet without need for a power cord. Most of these disseminate only one liquid.

It is advantageous to be able to disseminate two or more different liquids sequentially. For example, in the field of air fresheners, it is known that people become accustomed to a single fragrance (the art describes this as “habituated”) and it loses its effectiveness. Dissemination of a second, different fragrance after a suitable time can counter this. It may also be useful to disseminate entirely different liquids, for example, a fungicide, followed by a fragrance to mask the odour of the fungicide.

The problem of multiple liquids has been addressed by the art. One means is to provide a plurality of volatile materials on a rotatable plate or cartridge, in which the desired liquid is rotated into a dissemination position, that is, a position in which it is brought into juxtaposition with a disseminating means, such as a heat source or a forced current of air from a fan or an impeller. Typical examples of such devices are described in US published applications 2002/0068010 and 2004/0009103. While undoubtedly effective, such devices are large and mechanically complex, one drawback of which is that they can never be available as plug-in devices. Moreover, it is necessary to buy individual cartridges, with a restricted choice of fragrances and a quantity restricted by the size and nature of the cartridge.

Another proposed means is to provide stationary fragrances and direct an air current to the desired fragrance alone. This generally involves having a multiplicity of pathways, one per liquid, and blocking the pathways to all but the desired liquid. Typical examples include PCT published applications WO2003/028775 and WO2004/096300 and International applications PCT/CH2006/000011 and PCT/CH2006/000142. In the first two of these documents, the devices described are, by their nature, limited to two liquids. In the case of second two, this problem is overcome, but the devices are relatively bulky and not suitable for a small plug-in device.

It has now been found that it is possible to provide, in a compact, simple, inexpensive device, a plurality of volatile liquids that can be disseminated into an atmosphere in a regulated fashion. The invention therefore provides a device adapted to disseminate into an atmosphere in sequence a plurality of active volatile liquids, comprising a plurality of individual liquid reservoirs, a dissemination element associated with each reservoir, a central source of forced air current and one air channel per reservoir leading from an entrance at the central source to each dissemination element, air current being provided to one channel at a time by means of an air-tight rotatable barrier comprising a plurality of apertures, the spacing of these apertures being adapted to close all but one entrance at any given time.

The invention further provides a method of providing sequentially in an atmosphere a plurality of volatile liquids, comprising the steps of

(a) providing each of a plurality of volatile liquids in individual reservoirs with dissemination elements extending therefrom; and
(b) providing to the dissemination elements a forced air current from a central source of forced air current and one air channel per reservoir leading from an entrance at the central source to each dissemination element, air current being provided to one channel at a time by means of an air-tight rotatable barrier comprising a plurality of apertures, the spacing of these apertures being adapted to close all but one entrance at any given time.

The reservoir can be any suitable reservoir. It may be made of any suitable material or of any suitable size, shape or configuration, and the skilled person can easily determine suitable characteristics in each individual case. Typical materials include glass, metals, plastics, suitably lined paper and cardboard and ceramics. In one particular embodiment, the reservoir is made of a transparent or translucent material so that the liquid level can be seen, thus making it easy to determine when the liquid has been exhausted and the reservoir requires refilling or replacing. One of the advantages over the prior art is that, consistent with the number of reservoirs and overall desired size of the device, the reservoirs can be of a relatively large capacity in comparison with the capacities of the cartridges of the prior art. Moreover, they permit of a more versatile apparatus, as the user is no longer constrained to the liquids on a particular cartridge—the user can choose new liquids at any time. The reservoirs can be made available as refills for easy insertion into an existing apparatus. A typical refill will have the shape of a bottle of any suitable cross-section.

The dissemination element may be any element suitable for the conveyance of liquid from a reservoir to an atmosphere, at which point dissemination takes place because of an airflow impinging on the element. A typical dissemination element is a porous wick, of the type well known to the art and widely used in commercial air fresheners. Such wicks may be made of any suitable material, for example, porous plastics, compressed cellulosic material, dried plant materials, and sintered powdered metals or ceramics. The wicks may be cylindrical or they may be stamped out of a flat, porous material, such as porous cardboard. Another type of dissemination element is an external capillary emanator, that is, a surface or solid on the surface of which channels of capillary dimensions have been formed. This type of element has the advantage of avoiding the “fractionating” effect that some wick materials can have on fragrances that are complex mixtures. A typical example of a capillary emanator is described in U.S. Pat. No. 4,913,350.

The source of forced air current may be any suitable source. It may be, for example, a compressed gas stored in a bottle or aerosol and gradually released. It may also be a fan or impeller, which rotates and forces surrounding air to flow. The electrical power to drive such a fan may be supplied by any convenient means, such as batteries, solar cells and mains electricity. In the case of mains electricity, the device can comprise power pins for direct plugging into a power point and a transformer to reduce the mains voltage to a suitable level. The fans or impellers used are typically the small blowers used to cool laptop computers, and they are readily commercially available in a wide variety of sizes and types.

The source of forced air current is a central source, that it, it supplies air current to all of the dissemination elements and thus disseminates liquid into the atmosphere. To do this, there extend from the central source a plurality of individual channels leading to the individual dissemination elements. This may be provided on any convenient manner, a typical example being the provision of a chamber, the entrances to the channels being located on a wall or walls of this chamber. The chamber may contain the source of air current itself, for example, a blower, or it may be merely a plenum chamber that receives the air current from elsewhere in the device.

The allocation of air current to the individual channels is performed by an air-tight rotatable barrier having apertures. By “air-tight” is meant that the only means of escape of the air current from the device is through an aperture that is aligned with a channel. The rotation of the barrier may be achieved by any convenient means, but in a typical arrangement it is driven by a rotating element directly in contact therewith, for example, a rubber-faced driving wheel or a cogwheel meshing with corresponding teeth in the barrier.

One embodiment of this is the provision in the chamber hereinabove mentioned of a drum rotatable about its central axis and having on one side an entrance for air current and on its circumferential rim a plurality of apertures, which, in the course of rotation of the drum come into and move out of alignment with the entrances of channels leading to the individual dissemination elements. A variety of arrangements is possible, one example being a drum rotatable about an essentially horizontal axis, with the reservoirs, dissemination elements and channels arranged beneath it. This is, of course, not necessarily the case, but such an arrangement is relatively compact and is good for relatively small plug-in devices. The invention will hereinafter be described with reference to this particular embodiment, but the skilled person will appreciate that other variants are possible and lie within the scope of this invention.

The channels may be defined, and separated from other channels, by walls, which contact sufficiently closely the circumferential outer surface of the drum to inhibit air leakage from one channel to another, but not so closely that they inhibit rotational movement of the drum.

The arrangement ensuring that air is blown down only one channel at a time may be achieved in any convenient way, but it may be achieved by a simple arrangement of the components. In the following exemplary embodiment, the terms “circumferential width” and “circumferential distance” are used. These refer to distances measured along the circumference of the drum, including surfaces in contact with that circumference, which are considered for the purposes of this invention to be part of a circle of the same diameter. In the exemplary embodiment,

(a) the device comprises a drum rotating about a horizontal axis and having apertures in a circumferential surface, as hereinabove described;
(b) there is a plurality of channels, comprising a single duct within which individual channels are defined by sidewalls of the duct and intermediary walls placed therein, the intermediary walls contacting the circumferential surface in an air-tight fashion across the whole circumferential width of the wall, said circumferential width being greater than the width of an aperture; and
(c) the circumferential distance from an edge of an aperture to the corresponding edge of an adjacent aperture is at least equal to the circumferential distance between the sidewalls.

Thus, in such an arrangement, only one aperture at a time corresponds with the entrance of a channel and with only one channel at a time. This aperture will correspond sequentially with all channels before the next aperture in line on the circumferential encounters the first channel. The smaller the circumferential distance between the sidewalls of the duct, the larger the number of apertures possible. This number will depend entirely on preferences and needs, but in a typical compact arrangement, there will be 3-4 apertures.

Such a simple mechanical arrangement is easily realised and inexpensive, making it particularly suitable for a plug-in device.

The devices according to this invention can be easily and cheaply made form known materials, such as metal, glass, ceramics or plastics, plastics being especially convenient, as all the necessary parts can be injection-moulded. Moreover, they are both reliable and effective in use.

The invention will now be further described with reference to the drawings, which depict a preferred embodiment and which are not intended to be in any limiting on the scope of the invention. This particular embodiment is a plug-in air freshener and disseminates three fragrances in sequence.

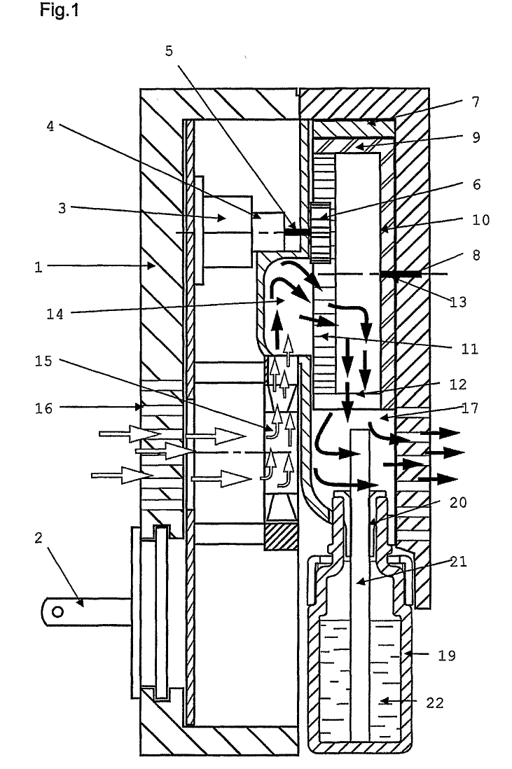

FIG. 1 depicts a front-to-back vertical cross-section through a device.

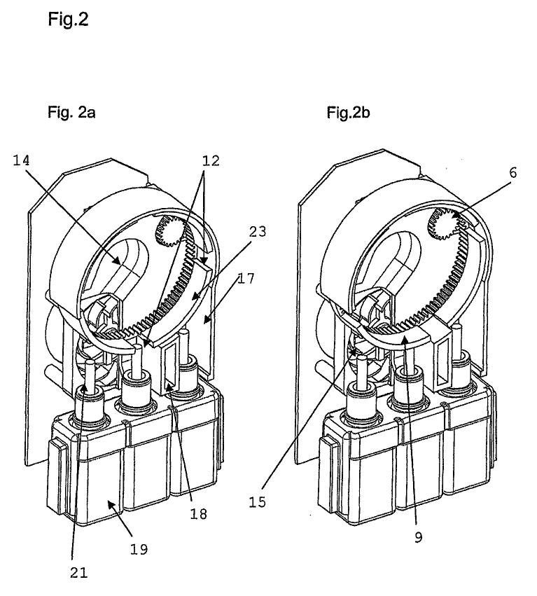

FIG. 2 depicts two schematic part-section perspective views of the embodiment of FIG. 1, each one in a different configuration.

A device comprises a housing 1 which comprises pins 2 suitable for plugging into a domestic electrical power socket. These provide the power source 2 drives a primary motor 3, which is attached to a gearbox 4 that suitably reduces the rotational speed of the motor. Extending from the gearbox 4 is a spindle 5 on which is a cog wheel 6. This meshes with corresponding gear teeth arranged around the internal circumference of a drum 9 that is rotatably mounted on a shaft 8, the rotation of the cog wheel 6 thus rotating the drum within a cylindrical cavity 7 in the device. There are additionally guide wheels (not shown) mounted on the sides of the cylindrical cavity that help the rotation of the drum.

The drum 9 has one closed end 10 and one open end 11, the gear teeth hereinabove mentioned being located around the circumference of the open end. Through this open end can come a flow of air (black arrows), forced by a blower 15, which takes the air from a rear vent 16 (white arrows) and expels it through an internal vent 14 into the drum through the open end 11.

Evenly spaced around the circumference of the drum is a series of apertures 12. These interface with the entrances of air channels 17, each of which channels leads to a wick 21, which is mounted in a plastic insert 20 in a reservoir 19 containing a volatile liquid 22.

There are three channels 17, essentially provided by dividing a single duct with intermediate walls 18 (these can be seen in FIG. 2—the wall portion between the left-hand and middle wicks is not shown for clarity). The width of an intermediate wall, measured along the circumferential contact with the drum, is slightly wider than the width of an individual aperture. That portion of the drum circumference between adjacent apertures, designated as 23 in FIG. 2a, is of such length that only one aperture 12 can correspond to any channel 17 at any time.

Thus, in operation, as the drum 9 rotates (anti-clockwise in FIG. 2), an aperture 12 will correspond first with the left-hand channel of the three channels 17 in the particular embodiment. Air from the blower 15 and passing through the internal vent 14 into the interior of the drum will go down this channel and convey liquid from the left-hand wick 21 into the atmosphere. As rotation continues, the aperture will encounter the first wall 18 (not shown in FIG. 2, but the encounter of the aperture with the right-hand wall is shown in FIG. 2b, and it will be the same). As the surface of the wall in contact with the drum is wider than the aperture, no air flow takes place at this point, and the air is vented through a relief vent (not shown).

The aperture then moves into correspondence with the middle channel, and air blows down that channel to convey the liquid of that particular wick into the atmosphere. At this point, the adjacent aperture does not correspond with any channel. The aperture then encounters the second wall 18 and the same thing as previously described happens, there is no air flow through any channel. Air flow resumes when the aperture encounters the right-hand channel 17. Finally, the aperture rotates such that the aperture corresponds to the circumferential surface of the cylindrical cavity 7, and no further air escapes through that channel. At this point, the following aperture 12 has not yet come into correspondence with the left-hand channel, so no air flows and no liquid is disseminated.

The skilled person will realise that the quantity and timing of liquid dissemination can be regulated by selecting at least one of the speed of rotation of the drum, the speed of the air flow and the relative dimensions of the components. This is well within the ordinary skill of the art.

The above-described embodiment is an example only and the skilled person will be able to realise many other ways of performing the invention not described herein, but lying within the scope of the invention.

1. A device adapted to disseminate into an atmosphere in sequence a plurality of active volatile liquids, comprising a plurality of individual liquid reservoirs, a dissemination element associated with each reservoir, a central source of forced air current and one air channel per reservoir leading from an entrance at the central source to each dissemination element, air current being provided to one channel at a time by means of an air-tight rotatable barrier comprising a plurality of apertures, the spacing of these apertures being adapted to close all but one entrance at any given time.

2. A device according to claim 1, in which the dissemination element is chosen from a porous wick and an external capillary emanator.

3. A device according to claim 1, in which the forced air current is provided by a blower.

4. A device according to claim 1, in which the rotatable barrier has the form of a drum, open at one end and comprising apertures on its circumferential edge.

5. A device according to claim 4, in which
(a) the drum rotates about a horizontal axis;
(b) there is a plurality of channels, comprising a single duct within which individual channels are defined by sidewalls of the duct and intermediary walls placed therein, the intermediary walls contacting the circumferential surface in an air-tight fashion across the whole circumferential width of the wall, said circumferential width being greater than the width of an aperture; and
(c) the circumferential distance from an edge of an aperture to the corresponding edge of an adjacent aperture is at least equal to the circumferential distance between the sidewalls.

6. A device according to claim 1, in which the device is a plug-in device.

7. A method of providing sequentially in an atmosphere a plurality of volatile liquids, comprising the steps of
(a) providing each of a plurality of volatile liquids in individual reservoirs with dissemination elements extending therefrom; and
(b) providing to the dissemination elements a forced air current from a central source of forced air current and one air channel per reservoir leading from an entrance at the central source to each dissemination element, air current being provided to one channel at a time by means of an air-tight rotatable barrier comprising a plurality of apertures, the spacing of these apertures being adapted to close all but one entrance at any given time.
